(19) United States
(12) Patent Application Publication
(10) Pub. No.: US 2012/0100576 A1
(43) Pub. Date: Apr. 26, 2012

(54) PERFUSION BIOREACTOR

(75) Inventors: Steffen Goletz, Berlin (DE); Rainer Stahn, Berlin (DE); Annett Hillemann, Berlin (DE)

(73) Assignee: GLYCOTOPE GMBH, Berlin (DE)

(21) Appl. No.: 13/382,763

(22) PCT Filed: Jul. 8, 2010
(86) PCT No.: PCT/EP2010/004177

§ 371 Date: Jan. 6, 2012

Related U.S. Application Data
(60) Provisional application No. 61/223,933, filed on Jul. 8, 2009.

(51) Int. Cl. C12P 21/04; C12P 1/04; C12P 1/02; C12N 7/04; C12M 1/00; C12N 1/00; C12N 1/14; C12M 1/34; C12M 3/00; C12M 1/12; C12P 19/34; C12N 5/02

(52) U.S. Cl. 435/70.1; 435/91.1; 435/170; 435/171; 435/236; 435/383; 435/252.1; 435/255.1; 435/287.1; 435/289.1; 435/297.2; 435/303.1

(57) ABSTRACT

The present invention pertains to a system for culturing cells comprising a culturing bag and a continuous flow centrifuge wherein the cells are continuously separated from the supernatant and are recycled into the culturing bag. Further provided are methods for culturing cells and for producing a biological substance using the device for culturing cells, and the use of a bag for culturing cells in said device or said methods. In particular, a perfusion system for culturing cells is provided wherein the wave technology for culturing cells is combined with continuous flow centrifugation for separating the medium from the cells.

### FIELD OF THE INVENTION

The present invention pertains to a system for culturing cells comprising a culturing bag and a continuous flow centrifuge wherein the cells are continuously separated from the supernatant and are recycled into the culturing bag. Further provided are methods for culturing cells and for producing a biological substance using the system for culturing cells, and the use of a rigid or flexible bag for culturing cells in said device or said methods.

### BACKGROUND OF THE INVENTION

In vitro cell culture is an important operation for obtaining cell products. In particular, cell culture may be used for producing pharmaceuticals such as antibodies, cytokines, enzymes, viral gene vectors and viruses for vaccination. Methods for culturing cells can be divided into two major categories: On the one hand, batch systems are used. In batch systems the cell culture is allowed to grow to a point at which the desired component is believed to be at optimal concentration. Then the entire vessel is harvested to separate the cells from the medium containing the secreted products. This separation is typically done by filtration or centrifugation. On the other hand, perfusion bioreactors are used. In a perfusion bioreactor, at some point after culture inoculation, the liquid media is circulated out of the bioreactor through a separation device and then returned to the bioreactor. The separation device is typically a filtration device or settling device. The separation device selectively removes a percentage of the contents, including any secreted product and waste product, of the liquid stream from the bioreactor. The volume removed is replaced in the bioreactor with growth medium. In these types of systems, separation can occur for a period of time as long as wastes are removed and the culture medium is replenished.

However, the devices used for these batch or perfusion systems are quite complex and require frequented sterilisation of their parts before use and thoroughly cleaning after use. This adds to the overall cost and reduces the efficiency of the cell culture production and filtration process.

The recent technology for cell culture systems uses rigid or flexible bags as vessels for the cell culture. These bags are placed on a platform which tills to one side and the other and thereby induces a wave motion in the cell culture. These cell culture bags are inexpensive in their production and can be discarded after use.

It is the object of the present invention to provide an improved cell culturing system. It is a further object to provide an improved method for producing a biological substance using a cell culture.

### SUMMARY OF THE INVENTION

Now, according to the invention, a disposable cell culture bag is used in a perfusion system for culturing cells together with a continuous flow centrifuge for continuously or periodically removing at least a part of the medium from the cell culture. It was surprisingly found that the combination of these two systems leads to an increase in the production yield while maintaining or even improving the quality of the products produced by the cell culture.

Accordingly, in a first aspect the present invention provides a system for culturing cells, comprising:
a) a bag for containing a cell culture comprising at least one outlet and at least one inlet; and
b) a continuous flow centrifuge for separating cell culture into a first fluid of decreased cell density and a second fluid of increased cell density comprising at least one inlet, a first outlet for the first fluid, and a second outlet for the second fluid.

Furthermore, in a second aspect, the present invention provides a method for cultivating cells comprising the steps of:
a) providing a cell culture in a bag;
b) transferring a part of the cell culture from the bag to a continuous flow centrifuge;
c) separating said part of the cell culture into a first fluid of decreased cell density and a second fluid of increased cell density by continuous flow centrifugation; and
d) returning at least a part of the first and/or second fluid, preferably the second fluid, to the cell culture in the bag.

The present invention further provides, in a third aspect, a method for producing a biological substance comprising the steps of:
a) providing a culture of cells capable of producing the biological substance in a bag;
b) transferring a part of the cell culture from the bag to a continuous flow centrifuge;
c) separating said part of the cell culture into a first fluid of decreased cell density and a second fluid of increased cell density by continuous flow centrifugation; and
d) obtaining the biological substance from the first and/or second fluid; and
e) returning at least part of the first and/or the second fluid to the cell culture in the bag.

In a fourth aspect, the present invention provides the use of a culturing bag comprising at least one outlet and at least one inlet for cultivating cells in a device according to the first aspect of the present invention or in a method according to the second or third aspect of the present invention.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

The present invention provides a system, preferably a device for culturing cells comprising a bag for containing a cell culture connected in a circular system to a continuous flow centrifuge. Using such a perfusion system for culturing cells, a part of the cell culture can continuously or periodically be transferred from the bag to the continuous flow centrifuge wherein a part of the culture medium is separated from the cell culture thereby obtaining a first fluid of decreased cell density and a second fluid of increased cell density. At least a part of the first and/or second fluid, preferably the second fluid wherein the cell density is enriched is recycled into the culturing bag.

The system can be used for culturing cells e.g. for producing cells or for producing a biological substance by the cells. Thus, the withdrawn culture medium also may contain the biological products of interest which are produced by the cells and are e.g. secreted into the medium. These desired biological products can be easily and continuously isolated from the cell culture that is processed in the continuous flow centrifuge. The same applies in case the cell culture is used for obtaining cells. Thus, the system according to the present invention allows the continuous harvesting of the desired products (either cells or substances produced by the cells) in a circular system. By constantly withdrawing a part of the culture medium in the technology according to the present invention, waste and degradation products of the cell culture may also be removed.

It was surprisingly found that the system according to the present invention which combines elements of a perfusion system with a continuous flow centrifuge has significant advantages over known systems. E.g. the cell growth rates and in particular the production yield in case the cell culture system according to the invention is used for producing a biological product is remarkably increased. Compared to conventional bag systems the yield was increased by 50 -100%. Furthermore, the yield was also remarkably increased by 50%-150% compared to conventional fermenter systems. Besides an improved yield, it was also found that the obtained biological substances produced by the cells cultured with the system according to the present invention may also show improved properties such as e.g. an improved homogeneity e.g. with respect to their glycosylation profile. In particular, the stability and quality of the obtained biological substances produced by the cells cultured with the system according to the present invention was found to be at least similar to that obtained by conventional culturing systems, despite the highly improved productivity. Therefore, the system according to the present invention provides important advantages compared to the prior art.

For maintenances of the cell culture, fresh medium containing nutrients required by the cells can be added to the cell culture. Thereby, also the volume lost by withdrawing the culture medium separated by centrifugation may be replaced.

As described above, in the system for culturing cells according to the invention, a part of the medium of the cell culture is preferably constantly or periodically withdrawn and processed in the continuous flow centrifuge. The withdrawn cell culture medium is preferably replaced by fresh medium and the cell density preferably is held at a constant level thereby preferably maintaining the cells in the growth phase. Thus, the overall conditions in the cell culture are held constant, preferably at optimal conditions for cellular production and survival.

In a preferred embodiment, a part of the cellularly enriched cell culture obtained after centrifugation as second fluid of increased cell density is removed from the system before the remaining part of the cellularly enriched cell culture is recycled to the culturing bag. In this embodiment, a certain amount of cells is constantly withdrawn from the system (also referred to as “bleeding”). However, at the same time the cells of the cell culture continue to divide, thereby increasing the number of cells in the cell culture. If cellular growth and withdrawal of cells is held at a balance, the overall cell density in the cell culture can be maintained at a constant level. Furthermore, in one embodiment the cells are kept at particularly high density preferably with a high viability, which may be achieved by provision of fresh media and/or media components. Furthermore, according to one embodiment the cells in the cell culture preferably do not reach a stationary phase, but rather will remain in the growth phase during the entire culturing process. Compared to cells in the stationary phase, cells in the growth phase are less prone to reducing cell production and dieing. This embodiment is particularly suitable in case cells are cultured as host cells which show the best production rates in the growth phase.

Due to the possibility of providing optimal conditions during the entire culturing process, the yield of a desired secreted product could be surprisingly increased by up to 100% compared to conventional perfusion systems. The system and methods according to the invention are in particular beneficial for producing biological products, in particular recombinant products. By the possibility to maintain the conditions of the cell culture in a desired, optimal range, in particular the production of biological substances which are otherwise difficult to obtain in a suitable quality and quantity is considerably improved. For example, glycosylated proteins carrying a specific, desired glycosylation pattern can be obtained with high yield and with the same or even a more homogeneous glycosylation, the same or even less degradation products (protein and glycosylation) using the device and methods of the present invention.

The technology of the present invention is subsequently described in further detail.

a) The System for Culturing Cells

In a first aspect, the present invention provides a system for culturing cells comprising a bag for containing a cell culture and a continuous flow centrifuge. The culturing bag comprises at least one outlet which is connected (directly or indirectly) to at least one inlet of the centrifuge. Using this connection, cell culture can be continuously or periodically transferred from the bag to the centrifuge. In the centrifuge, the cell culture can be separated into a first fluid and a second fluid wherein the cell density is decreased in said first fluid and increased in said second fluid compared to the cell culture in the culturing bag. The continuous flow centrifuge contains a first outlet through which the first fluid leaves the centrifuge. Furthermore, the centrifuge comprises a second outlet through which the second fluid leaves the centrifuge.

According to one embodiment the outlet of the bag is connected (directly or indirectly) with the inlet of the centrifuge and at least one outlet of the continuous flow centrifuge is connected with the inlet of the culturing bag. Several ways are, feasible for establishing a respective connection, including the use of adapters and further intermediate devices. According to this embodiment, means are provided for returning at least a portion of the first and/or second fluid, preferably the second fluid, to the cell culture after separation by the continuous flow centrifuge, e.g. by using appropriate connections and or tubings.

According to a preferred embodiment, the second fluid obtained by centrifugation is at least partially recycled respectively returned through the connection to the cell culture in the culturing bag. Thus, the present invention provides a system for culturing cells comprising a culturing bag and a continuous flow centrifuge wherein the cells are continuously separated from the supernatant and are recycled into the culturing bag. A preferred embodiment of the system for culturing cells according to the present invention is shown in FIG. 1. In another embodiment, the first outlet of the centrifuge is connected to the inlet of the bag. In this embodiment, at least a portion of the first fluid of decreased cell density is transferred back into the bag.

The following embodiments in particular refer to the preferred embodiment wherein the second outlet of the continuous flow centrifuge is connected to the inlet of the bag, enabling a recirculation of at least a part of the second fluid of increased cell density obtained after centrifugation. However, the embodiments and features described below may also apply, as appropriate, to other embodiments wherein, for example, the first outlet of the centrifuge is connected with the inlet of the bag, enabling a recirculation of at least a part of the first fluid of decreased cell density obtained after centrifugation, or wherein none of the outlets of the centrifuge are connected with an inlet of the bag. Suitable modifications of said embodiments and features which are necessary to match these other embodiments of the system according to the invention are included herein and can be readily derived from the following description by a person skilled in the art.

In a preferred embodiment of the first aspect of the invention, a system for culturing cells is provided, comprising:
a) a bag for containing a cell culture comprising at least one outlet and at least one inlet; and
b) a continuous flow centrifuge for separating cell culture into a first fluid of decreased cell density and a second fluid of increased cell density comprising at least one inlet, a first outlet for the first fluid, and a second outlet for the second fluid

wherein at least one outlet of the bag is connected with at least one inlet of the centrifuge and at least one outlet of the continuous flow centrifuge is connected with at least one inlet of the bag.

For continued cell growth and cell survival, it is beneficial to provide the cell culture with a sufficient amount of oxygen. The oxygen normally is provided as gas and has to reach the cells in the liquid cell culture. To obtain an adequate supply of oxygen in the entire cell culture, the culture is preferably mixed. First, this mixing is provided by the circular flow of the cell culture from the bag to the centrifuge and back to the bag. However, a better mixing may be obtained by additionally agitating the cell culture in the culturing bag. To this end, the culturing bag is placed, in a preferred embodiment, onto a platform which is capable of moving the bag in a seesaw, rocking or rotational motion. Due to this motion, waves are induced in the cell culture. The platform may be movable in only a single degree or in two degrees of freedom. Furthermore, the platform may be tilted in the seesaw or rocking motion through an angle in the range of 1 to 45 degrees, preferably 1 to 12 degrees from the horizontal position. Preferably, the platform is tilted in an angle of up to 10 degrees, more preferably up to 8 degrees, and most preferably up to 6 degrees. The preferred rate of the seesaw, rocking or rotational motion is in the range of 1 to 300 movement cycles per minute, preferably 1 to 60 movement cycles, more preferably 1 to 30 or 1 to 20 movement cycles and most preferably 1 to 12 movement cycles per minute. To secure the bag from falling off the platform, the bag may be fixed on the platform using restraining straps and/or adhesives. Alternatively or additionally, the platform may be equipped with a rigid box, barrel or cylinder where the bag fits in. The platform may be moved, for example, pneumatically, hydraulically or electrically.

For transferring cell culture from the culturing bag to the centrifuge, the at least one outlet of the bag preferably is located beneath the surface of the cell culture. Thereby, the centrifuge is prevented from drawing air which may be detrimental to the operation of the centrifuge. Several different means may be employed to ensure that the outlet of the bag is located beneath the surface of the cell culture. For example, the outlet may be located at the bottom of the bag, preferably in the middle part of the bottom of the bag, or it may be located in the lower part of the side of the bag, in particular near the bottom (see FIG. 3A).

Furthermore, the outlet of the bag may comprise a rigid or flexible tube which extends into the cell culture (see FIG. 3B). This tube should be designed such that it extends to the bottom or almost to the bottom of the culturing bag or is permanently submersed in the cell culture media. The flexible tube may additionally contain a weight at its end which ensures that the ending of the tube is always located at the bottom of the bag. Alternatively, the outlet of the bag may comprise a flexible tube extending into the interior of the bag at which end a floating device is attached (see FIG. 3C). During operation of the bag, the floating device floats on the surface of the cell culture. The tube connected to the floating device should preferably be long enough so that the floating device is capable of remaining on the surface of the cell culture irrespective of the amount of cell culture present in the bag and the degree of movement of the cell culture. A further tube may be attached to the bottom of the floating device or the tube may protrude through the floating device thereby protruding, respectively extending into the cell culture so that the part of the cell culture is not only withdrawn directly from the surface of the cell culture using said tube but is also taken from the lower layers of the cell culture. Also other variations are within the scope of the present invention, e.g. the tube may also be attached to the floating device.

As further means to prevent the centrifuge from drawing air, in a preferred embodiment, the device for culturing cells further comprises a means for forming a cell culture reservoir. Preferably, the cell culture reservoir is formed inside the culturing bag. The reservoir may be formed by providing one or more barriers at the bottom of the culturing bag. These barriers may be located inside the bag attached to or extending from the bottom of the bag (see FIG. 4A). The barrier thereby defines a separate basin respectively reservoir at the bottom of the bag wherein a part of the cell culture will remain preferably even during the entire movement cycle of the platform. Alternatively, the at least one barrier may be located on respectively form part of the platform onto which the bag is placed (see FIG. 4B). When a flexible culturing bag is placed on top of the platform, the barrier of the platform will dent the bag, thereby again forming a separate basin at the bottom of the bag. The bag or the platform may contain/exhibit one or more barriers, forming one or more separate cell culture reservoirs. When the bag comprises the cell culture medium, at least a portion of the cell culture medium is retained in said cell culture reservoir even if the culturing bag is moved, e.g. in a wave-like movement. The culture reservoir formed by the at least one barrier or barriers may preferably be located at one or both ends of the bag or in the middle of the bag. The embodiments described involving means for forming a cell culture reservoir, in particular at least one barrier, are also beneficial in order to increase the circulation of the cell culture medium within the bag. Thus, these elements can also be used without a continuous flow centrifuge. Thus, in one aspect the present invention also provides a culturing bag, which comprises means for forming a cell culture reservoir inside said culturing bag. According to one embodiment, said cell culture reservoir is formed by providing one or more barriers at the bottom of the culturing bag. According to one embodiment, the one or more barriers are attached to and/or protrude from the bottom of the bag. The bag and the one or more barriers may be formed as one piece or as connected pieces. Preferably, said one or more barriers define a separate basin, respectively reservoir at the bottom of the bag wherein a part of the cell culture will remain even if the culturing bag is moved in a seesaw, rocking or rotational movement during cultivation. Furthermore, the culturing bag may have one or more of the characteristics described herein in conjunction with the different aspects according to the present invention. It is referred to the respective disclosure. In particular, it may comprise at least one outlet and at least one inlet, wherein at least one outlet may preferably be located in the area of the culturing bag which forms the cell culture reservoir. Said outlet is positioned such that it is located underneath the surface of the cell culture medium when said cell culture reservoir comprises cell culture medium. As discussed above, this decreases the risk of drawing air when cell culture medium is removed through said outlet. According to one embodiment, a system for culturing cells is provided, which comprises a cell culturing bag and a platform for moving the culturing bag, preferably in a seesaw, rocking or rotational movement during cultivation. According to one embodiment, said system comprises means for forming a cell culture reservoir inside the culturing bag. According to one embodiment, a culturing bag as described in this paragraph is used in said system which comprises one or more barriers for forming the cell culture reservoir. As discussed above, by using a respective culturing bag the risk of drawing air through the at least one outlet of the culturing bag that is located within the area of the cell culturing reservoir is considerably reduced, respectively prevented. Said culturing bag may have the further characteristics of the culturing bag described herein in conjunction with the different aspects of the present invention. According to one embodiment, the platform comprises one or more barriers which form a cell culturing reservoir when the preferably flexible culturing bag is placed on the platform. The platform and the one or more barriers may be provided as one piece or separate, but attached pieced. As described above, the one or more barriers of the platform dents, respectively forms the culturing bag over said barrier(s), thereby providing a cell culturing reservoir within the culturing bag. Further details on said embodiments and characteristics of the cell culture reservoir are described above; it is referred to the above disclosure which also applies here.

Furthermore, the platform, which can be used in conjunction with the methods according to the present invention or the system described herein, may comprise a depression. When a flexible bag containing the cell culture is placed on top of such a platform, the bag will extend into the depression of the platform, thereby forming a cell culture reservoir (see FIG. 4D). The depression may be formed like a trench which preferably is long enough to cross the entire bottom of the bag. The platform may comprise one or more depressions and the depressions are preferably located in the middle or at one or both ends of the area of the platform covered by the bag. The depression can also be a concave depression with a round or otherwise formed outer line.

If a cell culture reservoir is present inside the culturing bag, at least one outlet of the bag is preferably located inside the cell culture reservoir or is in contact with the cell culture reservoir, for example via a tube through the first outlet. The tube and the outlet may also form one piece.

In one embodiment, the system for culturing cells according to the invention further comprises a cell culture reservoir which is positioned between the outlet of the bag and the inlet of the centrifuge. For example, the outlet of the bag may be connected to a vessel respectively container which provides, respectively contains the cell culture reservoir. The vessel further is connected to the inlet of the centrifuge. This embodiment is an example of an indirect connection between the bag and the continuous flow centrifuge. During the rocking movements, cell culture medium enters through the first outlet into the vessel which provides the cell culture reservoir. Said vessel has an outlet that is connected to the centrifuge. The outlet of the vessel used for transferring cell culture to the centrifuge is preferably located beneath the surface of the cell culture reservoir (see FIG. 4C). Thereby, the centrifuge is prevented from drawing air. Means as described above for the outlet of the bag may be used to ensure that the outlet of the vessel containing the cell culture reservoir which is used for connecting the vessel to the centrifuge is located beneath the surface of the cell culture reservoir. It is referred to the above disclosure which also applies here.

The system according to the invention may comprise one or more of the cell culture reservoirs described above. In particular, it may comprise more than one cell culture reservoir at different positions inside the bag, for example at opposed ends of the bag. Furthermore, it may comprise one or more cell culture reservoirs inside the bag and at least one cell culture reservoir outside of the bag, between the outlet of the bag and the inlet of the centrifuge.

The bag, respectively culturing bag used in the device for culturing cells according to the invention and/or the methods described herein may be any disposable container capable of receiving liquid media such as a rigid or flexible bag, preferably a collapsible bag. Preferably, it is a plastic bag, more preferably a thermoplastic bag. In a preferred embodiment, the culturing bag has only one single hollow interior space. Preferably, it does not contain a membrane and/or a stirring device. Besides the outlet and the inlet which are connected to the continuous flow centrifuge, the culturing bag may contain further connections. For example, the bag may contain a further inlet and a further outlet for introducing and withdrawing gas, respectively (see FIG. 2). These inlet and outlet ports may be used to provide the cell culture which oxygen and/or carbon dioxide and to withdraw gaseous waist from the cell culture. These further inlet and outlet ports may be equipped with filters to prevent the cell culture from being contaminated and from contaminating the environment. Furthermore, the bag may comprise a further inlet for introducing media, nutrients and/or inoculum into the bag. Additionally, the bag may also contain a further outlet for withdrawing cell culture, said further outlet not being connected to the continuous flow centrifuge. Moreover, the bag may contain further inlets and outlets or ports as desired, for example outlets for taking samples, separate inlets for introducing cells into the bag, and/or ports for connecting measuring devices. Additionally, the bag may contain more than one of the inlets, outlets and ports described above. In particular, the bag may comprise two, three, four or more outlets and/or two, three, four or more inlets which are connected (directly or indirectly) with one or more continuous flow centrifuges.

In one embodiment, the bag comprises at least two outlets for withdrawing cell culture which are connected to the same or different continuous flow centrifuges. These outlets are preferably located at opposite sides of the bag in such a manner that when the bag is tilted by the platform to one side, the first of these outlets is located beneath the surface of the cell culture, and when the bag is tilted to the other side, the second of these outlets is located beneath the surface of the cell culture. Preferably, cell culture is only drawn from that outlet which at that moment is located beneath the surface of the cell culture. This may be achieved, for example, by the use of valves or by controlling the suction of the centrifuge(s). Preferably, these controlling is performed automatically and more preferably is linked to and/or synchronized with the motions of the platform. Furthermore, these two or more outlets may be connected to the same or different cell culture reservoirs as described above. In case of cell culture reservoirs inside the bag, they are preferably connected to different reservoirs while in case of reservoirs outside of the bag, they may be connected to the same or to separate reservoirs.

In embodiments of the system according to the invention comprising a cell culture reservoir, the culturing bag may be specifically designed for forming said reservoir. In particular, the bag may contain one or more barriers for forming a cell culture reservoir as described above. Furthermore, the bag may comprise dents or protrusions fitting to the barriers or depressions, respectively, of the platform which can be used for forming a cell culture reservoir. However, according to one embodiment, the culturing bag fits to the barriers and/or depressions present in the platform due to the flexibility of said bag.

The culturing bag described herein, preferably in combination with the platform described herein, may not only be used in the device for culturing cells according to the invention but is also suitable for other uses. In particular, the bag, optionally in combination with the platform, may be used in other cell culturing devices. In particular, the present invention also generally discloses a culturing bag as described herein, preferably a culturing bag comprising one or more barriers as described herein or being designed for fitting to a platform having one or more barriers and/or one or more depressions for forming a cell culture reservoir as described herein.

The continuous flow centrifuge of the device for culturing cells according to the invention may be any continuous flow centrifuge suitable for separating cells from a liquid medium. According to the invention, a continuous flow centrifuge in particular is a centrifuge which can be fed with material to be separated and/or from which separated product can be withdrawn during an ongoing centrifugation process. In particular, material, in particular fluids, to be separated can be continuously introduced into the continuous flow centrifuge and both the heavy and the light phase produced by the centrifuge can be continuously withdrawn from the centrifuge during the centrifugation process. Preferably, a continuous flow centrifuge is capable of actively drawing material, in particular fluids, to be separated and/or actively discharging the separated products, in particular using built-in pumps. Suitable continuous flow centrifuges are commercially available, for example the Contifuge series from Heraeus and the CEPA centrifuges from New Brunswick Scientific. However, the continuous flow centrifuge used in the device according to the present invention should have two distinct outlets, one for a “light phase”, i.e. the first fluid of decreased cell density, and one for the “heavy phase”, i.e. the second fluid of increased cell density wherein the cells are enriched compared to the initial cell culture fluid entering the centrifuge. Preferably, the continuous flow centrifuge has one or more of the following characteristics: Obtainable g-values of at least 10 g, preferably at least 40 g, in particular between 10 g and 250 g, preferable about 40 g; maximal pump flow rates of at least 1 l/h, preferably at least 2 l/h, more preferably at least 4 l/h, in particular pump flow rates of between 0 and 10 litres per hour, preferable about 4 litres per hour; separation performance of at least 60% (i.e. 60% of the cells of the fluid entering the centrifuge are contained in the “heavy phase” after centrifugation at optimal conditions), preferably at least 80% (in particular for high density cell culture), more preferably at least 90%, in particular about 95%.

In certain embodiments, the system according to the invention comprises more than one continuous flow centrifuge, in particular, two, three or four continuous flow centrifuges. The inlets of the different centrifuges may be connected to the same or to different outlets of the bag. Furthermore, the outlets of the centrifuges for the second fluid of increased cell density (or first fluid of decreased cell density in less usual embodiments) may be connected to the same inlet of the bag or to several different inlets of the bag. Thus, each centrifuge of the system may be connected with the culturing bag in a separate circuit or two or more centrifuges may be arranged in a parallel manner in an interconnected or branched circuit. In another embodiment, two or more continuous flow centrifuges may be connected in a serial manner in one circuit, wherein one of the outlets of the first centrifuge is connected with the inlet of the next centrifuge. In order to improve cell recovery, the second centrifuge may be connected to the outlet for the first fluid of decreased cell density of the first centrifuge. Alternatively, for improving the yield of the supernatant, the second centrifuge may be connected to the outlet for the second fluid of increased cell density of the first centrifuge.

In the system for culturing cells according to the invention, the bag is connected to the continuous flow centrifuge. According to the invention, the term “connected to” in particular encompasses a direct as well as an indirect connection. In particular, it is referred to a fluid connection. For example, the bag is preferably in fluid connection with the centrifuge, that is, a fluid can be transferred from the bag to the centrifuge and/or from the centrifuge to the bag. The connection may be direct or it may be indirect, in particular comprising one or more adapters or further devices such as valves, pumps, vessels, collection reservoirs and the like between the two connected devices such as the bag and the centrifuge. A connection may connect two devices or more than two devices. It may be unidirectional, bidirectional or multidirectional, and preferably is unidirectional. If an outlet is connected to an inlet, then the connection preferably is unidirectional wherein the transfer is directed from the outlet to the inlet.

The bag and the centrifuge are preferably connected via tubing, more preferably via flexible tubing. Preferably, tubing is selected which can provide sterile conditions in the system, such as sterilizable tubing or disposable tubing. Furthermore, the tubing preferably is easy to clean. In particular, plastic tubing is preferred which is easy to clean and can be sterilized, if necessary. Alternatively, metal tubing may be used. The tube connecting the preferably second outlet of the centrifuge with the inlet of the bag may be connected to a means for introducing media, nutrients and/or inoculum. Such means may for example be a further inlet port connected to that tube. Alternatively or additionally, that means may be connected to the third inlet of the bag, if present.

In a preferred embodiment, the system for culturing cells according to the invention further comprises a means for withdrawing at least a part of the second fluid obtained after centrifugation. This means preferably is connected to the second outlet of the centrifuge or to the tube connecting the second outlet of the centrifuge with the inlet of the bag. The means for withdrawing a part of the second fluid may be connected to a device for further processing said second fluid or to a storage or waste tank for collecting the withdrawn second fluid. Furthermore, the system for culturing cells according to the invention may further comprise means for introducing gas into and/or withdrawing gas from the culturing bag. This means are preferably connected to a further (second) inlet and further (second) outlet of the bag, respectively. Furthermore, the system for culturing cells according to the invention may comprise a heating device, preferably a temperature-controlled heating device. The heating device preferably is attached to the platform. The heating device may be used to hold the cell culture at a desired temperature or in a desired temperature range.

In certain embodiments, the transfer of cell culture from the bag to the centrifuge is accomplished using a pump. The pump preferably is attached to the tube connecting the outlet of the bag with the inlet of the centrifuge. By controlling the pumping rate of said pump, the rate of the cell culture transfer from the bag to the centrifuge is controlled. Preferably, a peristaltic pump is used. Furthermore, the system may comprise a further pump for transferring the cell-enriched fluid obtained after centrifugation back into the culturing bag. However, in a particularly preferred embodiment, the continuous flow centrifuge already comprises one or more pumps which are suitable for performing the transport of the fluids from and to the culturing bag and to other destinations such as the removal of the first fluid from the system. In this embodiment, no additional pumps for transferring the cell culture are necessary and thus, the system preferably comprises no additional pumps for transferring fluids between the culturing bag and the centrifuge. However, the system may nevertheless comprise pumps, e.g. for transferring fresh medium, nutrients and/or inoculum into the bag, and/or introducing gas into or removing gas from the bag.

The system for culturing cells according to the invention may be equipped with one or more sensors. These sensors preferably measure one or more parameters selected from the group consisting of the cell density in the cell culture and/or the second fluid and/or the first fluid; the concentration of nutrients, oxygen, carbon dioxide, cell products, by-products, toxins, media components and/or cell degradation products and/or the pH value in the cell culture; the concentration of oxygen and/or carbon dioxide in the air in the bag; the concentration of cell products in the first fluid; the temperature of the cell culture; and the presence of cell culture at the first outlet of the bag and/or the presence of gas in the tube connecting the first outlet of the bag with the first inlet of the centrifuge. At least some of the sensors may be part of electronic feed-back loops controlling specific functions of the system such as the heating activity of the heating device, the rate of transfer of cell culture to the centrifuge, the rate of bleeding of the cell culture, and/or the rate of introduction of fresh medium, nutrients and/or gas containing oxygen and/or carbon dioxide.

IN a preferred embodiment, the system for culturing cells according to the invention has all of the features described in claims 1, 2, 3, 4 and 6 (i) to (ix) and the cell culture bag used therein has all features described in claim 5 (i) to (xi).

b) The Method for Culturing Cells

In a second aspect, the present invention provides a method for culturing cells comprising the following steps
a) providing a cell culture in a bag;
b) transferring a part of the cell culture from the bag to a continuous flow centrifuge;
c) separating said part of the cell culture into a first fluid of decreased cell density and a second fluid of increased cell density by continuous flow centrifugation; and
d) returning at least a part of the first and/or second fluid to the cell culture in the bag.

Preferably, a system for culturing cells according to the first aspect of the invention, including any of the embodiments of the device described above, is used in the method for culturing cells according to the invention. We refer to the above disclosure which also applies here.

The following embodiments in particular refer to the preferred embodiment wherein at least a part of the second fluid is recycled/returned to the cell culture in the culturing bag. However, the described embodiments and features may also apply, as appropriate, to other embodiments wherein, for example, in step d) at least a part of the first fluid is recycled to the cell culture in the culturing bag, or wherein neither the first nor the second fluid are recycled to the cell culture in the culturing bag. Suitable modifications of said embodiments and features which are necessary to match these other embodiments of the method for culturing cells according to the invention are included herein and can be readily derived from the following description by a person skilled in the art.

In the preferred embodiments of the second aspect of the invention, a method for cultivating cells comprising the steps of:
a) providing a cell culture in a bag;
b) transferring a part of the cell culture form the bag to a continuous flow centrifuge;
c) separating said part of the cell culture into a first fluid of decreased cell density and a second fluid of increased cell density by continuous flow centrifugation; and
d) recycling at least a part of the second fluid to the cell culture in the bag
   is provided.

In the first step, a cell culture in a bag is provided. The volume of the cell culture may preferably occupy 10 to 80% of the interior of the culturing bag, more preferably 10 to 50% or in particular 30 to 50%. Preferably, a bag as described above with respect to the system for culturing cells according to the invention is used in this step. It is referred to the above disclosure which also applies here. The cell culture may be provided by adding medium suitable for the cells to be cultured into the bag and inoculating the medium with an inoculum of the cells to be cultured. The medium may contain all the components required for viability and growth of the cells to be cultured or some or all of said components may separately be added into the bag. Components required by the cells to be cultured may include, for example, suitable carbon and nitrogen sources such as saccharides and/or amino acids which can be processed by the cells, growth factors, and antibiotics for selecting towards the desired cells. The medium and additional components needed for viability and growth of the desired cells are dependent of the cells to be cultured. However, those skilled in the art are capable of selecting the suitable medium and additives. After inoculating the medium with the desired cells, the cells are allowed to divide until a desired cell density is reached. Alternatively, the medium may be inoculated in a separate vessel and the inoculated medium may be transferred into the bag where cell growth occurs. In a further alternative, inoculation of the medium as well as cell growth up to a desired cell density are performed in a separate vessel and the final cell culture is transferred into the bag.

Using the method for culturing cells according to the present invention, any type of cells may be cultured. For example, the cells may be eukaryotic or prokaryotic and preferably are selected from the group consisting of bacteria, yeast, plant cells and animal cells. Specific examples of animal cells are insect cells, avian cells and mammalian cells such as rodent, duck, goose, primate or human cells. When using animal cells, the cells may be primary cells or cells of an established cell lines, in particular a human cell line. Preferably, immortalized cells are used. Most preferably, the cells are human cancer cells or cells derived therefrom or otherwise immortalized human cells, such as those immortalized by viral genes. Furthermore, hybridoma cells may be cultured in the system according to the invention, in particular hybridoma cells which produce antibodies. The cells may freely float in the cell culture (suspension cell culture) or they may be attached to the bottom and/or the side wall(s) of the bag (adherent cell culture). Furthermore, floating carrier molecules known to those skilled in the art may be used to create a suspension cell culture. However, using a suspension cell culture is preferred. Suspension cell cultures include those which contain cell lines which were made to become suspension cell lines or cell lines which de-adhere upon treatment or with certain media components. Preferably, the host cell is an immortalized human blood cell, preferably a host cell of myeloid leukaemia origin or any human myeloid or myeloid precursor cell or cell line which can be obtained from a leukaemia patient. For example, HEK293, KG1, K562, Mutz-2, Mutz-3, or PerC6 (see WO 97/00326 A1) cells may be used.

According to one embodiment, the human cell line is an immortalized human blood cell. Preferably, said immortalized human blood cell is a host cell of human myeloid leukaemia origin. This term particularly refers to any cell or cell line of human myeloid leukaemia origin, or any human myeloid or myeloid precursor cell or cell line which can be obtained from a leukaemia patient, or any myeloid or myeloid precursor cell or cell line which can be obtained from a human donor, or a cell or cell line derived from anyone of said host cells, or a mixture of cells or cell lines comprising at least one of those aforementioned cells. In a preferred embodiment of the invention the host cell of human myeloid leukaemia origin of the invention is the cell or cell line K562, KG1, MUTZ-3, NM-F9 [DSM ACC2606], NM-D4 [DSM ACC2605] or a cell or cell line derived from anyone of said host cells, or a mixture of cells or cell lines comprising at least one of those aforementioned cells. The host cell is preferably selected from the group consisting of NM-F9 [DSM ACC2606], NM-D4 [DSM ACC2605], NM-H9D8 [DSM ACC 2806], or NM-H9D8-E6 [DSM ACC 2807], or NM H9D8-E6Q12 [DSM ACC 2856], GT-2X [DSM ACC 2858] or a cell or cell line derived from anyone of said host cells, or a mixture of cells or cell lines comprising at least one of those aforementioned cells. Said cell lines are described in WO 2008/028686, herein incorporated by reference.

The method for culturing cells according to the invention may be used for producing cells of interest in large amounts. In particular, the method and the system of the invention are suitable for large scale and/or industrial production of cells. However, the method and system may also be used for small scale applications, in particular for laboratory use. In particular, the method for culturing cells according to the invention is capable of providing cells of good quality, in particular of high viability and/or with a high ratio of vital cells compared to dead or dying cells. If the cells produced by the method for culturing cells according to the invention are a product of interest, the method may be performed by continuously removing the second fluid of increased cell density obtained by centrifugation and thus the desired product from the system and optionally returning—if desired—the first fluid to the cell culture bag.

In preferred embodiments, the cells produce a biological substance of interest. The cells may produce the biological substance intrinsically or they may be modified to produce said biological substance, for example by recombinant technologies or viral infection, e.g. they may be transfected or transformed with a gene enabling the production of the biological substance or they may be infected with a virus or virus-like particle of interest, in particular one carrying a gene as described above. Furthermore, the cells may contain a selection marker which enables the distinction of the desired cells from other cells. For example, the selection marker may be an antibiotic resistance gene providing the cell with a resistance against a specific antibiotic. By adding that specific antibiotic to the culture medium, the cells of desire are selected for. Suitable recombinant techniques as well as selection markers are well known for the different cell types and therefore, need no specific description.

In preferred embodiments, the bag comprising the cell culture is placed on a platform as described for the system according to the invention. Using this platform, the bag and thus also the cell culture inside the bag is moved in a seesaw, rocking or rotational motion. The bag may be moved by the platform during the entire method for culturing cells or only during some time periods thereof. Furthermore, the movement of the platform may be controlled as described above.

In the second step of the method for cultivating cells according to the invention, a part of the cell culture is transferred from the bag to a continuous flow centrifuge. Suitable continuous flow centrifuges are described above; we refer to the above disclosure which also applies here. For transferring cell culture from the bag to the centrifuge, the bag comprises a first outlet which is connected to a first inlet of the centrifuge. This outlet of the bag preferably is connected to the inlet of the centrifuge by a tube, preferably a plastic or metal tube, more preferably a flexible plastic tube. In order to provide a trouble-free operation of the centrifuge, the centrifuge should be prevented from drawing air. To this end, the first outlet of the bag preferably is constantly in contact with the cell culture. Means for providing said constant contact are described above with respect to the device for culturing cells according to the invention. In particular, the outlet of the bag may be located at the bottom or near the bottom of the bag or may comprise a tube extending into the cell culture.

The amount of cell culture transferred from the bag to the centrifuge preferably is in the range of 0.1 to 100% of the total amount of cell culture in the bag per hour, more preferably 1 to 80%. In order to obtain a high throughput, the transfer from the bag may be adjusted to a high rate such as preferably in the range of 20 to 80%, more preferably about 60%. However, in order to lower the stress of the cells, the transfer rate may be adjusted to a lower rate such as preferably in the range of 1 to 30%, more preferably 1 to 10%, most preferably 2 to 5%.

Furthermore, as described above, a cell culture reservoir may be formed inside the bag, ensuring that the outlet of the bag will constantly be in contact with the cell culture. As described above, this reduces the risk of drawing air. As described above with respect to the device for culturing cells according to the invention, the cell culture reservoir may be formed using one or more barriers which may be present inside the bag or may be located on the surface of the platform but e.g. may also form an integral part thereof. As an additional effect, using such barrier(s) may result in a more thoroughly mixing of the cell culture while agitating the cell culture. Alternatively, the platform onto which the culturing bag is placed may comprise one or more depressions for forming the cell culture reservoir as described above. In another embodiment, the cell culture reservoir may be located outside of the bag. For example, the outlet of the bag may be connected to a further vessel, respectively container, containing respectively providing the cell culture reservoir. Said vessel comprises an outlet located beneath the surface of the cell culture reservoir, when said reservoir is filled with cell culture, which is connected to the first inlet of the centrifuge.

The transport of the part of the cell culture from the bag to the centrifuge may be driven by the suction power of the centrifuge, by gravitation or by a pump attached to the tube connecting the outlet of the bag with the inlet of the centrifuge. The pump preferably is a peristaltic pump. If the cell culture is transferred from the bag to the centrifuge using a pump, the rate of feeding cell culture to the centrifuge may be controlled by controlling the pumping rate of the pump. The cell culture is preferably transferred to the centrifuge in a continuous or periodic manner and/or at a rate and time as desired. The rate of the transfer of cell culture to the continuous flow centrifuge may be adjusted as required. Parameters which may be relevant for the suitable transfer rate include, for example, the separation performance of the centrifuge at the respective transfer rate, the growth rate of the cells, the production rate of the cells with respect to a desired biological substance, the occurrence or elimination of by-products and the rate of the further processing of the fluids obtained after centrifugal separation. In particular, the transfer rate of the cell culture from the bag to the centrifuge should be adjusted and/or controlled in such a manner that the centrifuge is prevented from drawing air. This may be achieved, for example, by controlling the suction of the centrifuge and/or the submersion of the bag's outlet in the cell culture and/or the presence or amount of cell culture in the reservoir, if present. In particular, said controlling may be performed automatically, wherein the necessary information is obtained by measuring devices and the suction of the centrifuge is controlled by an integrated circuit interpreting the measured information. Preferably, the parameters are selected in order to optimize the amount and quality of the biological product to be obtained.

In the third step of the method for cultivating cells according to the invention, the part of the cell culture transferred to the centrifuge is separated into a first fluid of decreased cell density and a second fluid of increased cell density by continuous flow centrifugation. The characteristics of the centrifugation should preferably be selected in view of one or more of the following parameters:
a. the amount of cells present in the first fluid;
b. the percentage of cells surviving the centrifugation procedure;
c. the degree of enrichment in cell density of the second fluid compared to the cell culture in the bag;
d. the time necessary for achieving the desired separation.

In particular, the amount of first fluid obtained by centrifugation should preferably be as high as possible while at the same time maintaining a good separation of cells from said first fluid and a high cell viability during centrifugation. However, the cell density in the second fluid should be low enough so that said second fluid can readily be transferred back into the bag if desired.

According to the invention, the first fluid of decreased cell density has a cell density that is lower than the cell density of the cell culture transferred to the centrifuge. Preferably, the cell density is decreased by a factor of 1.2, more preferably 1.5, 2, 3, 5, 10, 20, 50, 100, 200, 500 or 1000. Preferably, the first fluid of decreased cell density is substantially cell-free. In particular, the first fluid contains 1*10̂6 cells per millilitre or less, preferably 5*10̂5 cells per millilitre or less, more preferably 1*10̂5 cells per millilitre or less. Furthermore, according to the invention, the second fluid of increased cell density has a cell density that is higher than the cell density of the cell culture transferred to the centrifuge. Preferably, the cell density in the second fluid is increased at least 1.2-fold, more preferably at least 1.5-fold, 2-fold, 3-fold, 5-fold, 10-fold, 20-fold, 50-fold, 100-fold, 500-fold, and most preferably 1000-fold compared to the cell culture in the bag. Thus, depending on the obtained cell density, the second fluid of the present invention may also have a papescent consistency. The amount of the first fluid obtained after centrifugation preferably is at least 10% of the amount of cell culture entering the centrifuge, more preferably at least 20%, at least 30%, at least 40%, at least 50%, at least 60%, at least 70%, and most preferably at least 80%.

In the fourth step of the method for cultivating cells according to the invention, at least a part of the first and/or second fluid to is returned the cell culture in the bag. Preferably, at least a part of the second fluid obtained in the third step is recycled to the cell culture in the bag. The second fluid is transferred out of the centrifuge, through a second outlet of the centrifuge, and into the bag, through an inlet of the bag. The second outlet of the centrifuge may be connected to the inlet of the bag by a tube, preferably a plastic or metal tube, more preferably a flexible plastic tube. The transport of the second fluid may be driven by the output force of the centrifuge alone, by gravitation or by a pump attached to the tube connecting the second outlet of the centrifuge with the inlet of the bag. The pump preferably is a peristaltic pump.

In a preferred embodiment, a part of the second fluid of increased. cell density is removed from the system. Removing a part of the second fluid from the system is advantageous for the method for cultivating cells. By removing said second fluid, a certain amount of cells is withdrawn. This technique is also known as “bleeding” of the cell culture. However, at the same time, the cells in the cell culture divide, thus increasing the amount of cells. If the removal of the cells is adjusted so as to match the growth rate of the cells in the cell culture, the overall cell density in the cell culture can be maintained at a certain constant level or in a certain desired range. Without bleeding of the cell culture, the cells would grow and the cell density would increase until reaching a plateau. Then, the cells might switch from the growth phase into a stationary phase until eventually they would die. Using the bleeding technology, cell densities can be controlled and used for example to hold the cells in an optimal growth rate. Since for some cell types cellular production and cell viability is superior during the growth phase compared to the stationary phase, it is highly desirable to keep the cells in the growth phase. Thus, the second fluid, respectively a portion of the second fluid, preferably is removed from the system in an amount suitable to maintain the cells of the cell culture in the growth phase. In another embodiment, cells may be used which cellular production is highest in the stationary phase. Here, bleeding can be used to prevent the cell density from becoming too high so that cell viability is severely reduced. However, most of the cells are preferably held in the stationary phase in this embodiment. In particular, the amount of second fluid withdrawn for bleeding preferably is adjusted so as to maintain the cell density of the cell culture at a constant level or in a desired region, especially a level or region where cellular productivity is optimal.

To effect bleeding, a means for withdrawing said part of the second fluid may be attached to the second outlet of the centrifuge or to the tube connecting the second outlet of the centrifuge with the first inlet of the bag. Preferably, a valve is attached to said outlet or tube suitable for directing the flow of the second fluid either back to the bag or in another direction. The part of the second fluid withdrawn from the system may be subjected to further processing steps, stored in a storage tank, or discarded. Preferably, about 1 to 10% of the second fluid is withdrawn by bleeding. Alternatively, also other ways of bleeding may be used. For example, the separation performance of the continuous flow centrifuge may be adjusted so that the first fluid of decreased cell density which is withdrawn from the system contains an amount of cells suitable for effecting bleeding.

During the method for cultivating cells according to the invention, medium and/or nutrients may be added to the cell culture in the bag. Preferably, the nutrients and medium are added to the cell culture in an amount to provide the cells of the cell culture with sufficient fresh medium and/or nutrients, in particular sufficient to maintain the cells, i.e. to enable cell growth, viability and cellular production. The amount and/or type of medium and/or nutrients added to the culture may vary during the culturing process and in particular may depend on the specific phases of the culturing process, such as the initial growth of the cells, the production of a biological substance of desire, or others. Furthermore, the amount of medium and/or nutrients added to the cell culture may be similar to the amount of the first fluid withdrawn from the system after centrifugation, optionally added by the amount of second fluid withdrawn during bleeding of the cell culture. The volume added to the cell culture preferably is adjusted so as to maintain the volume of the cell culture in the system at a constant level or in a desired region.

Furthermore, gas containing oxygen and/or carbon dioxide may be introduced into the bag during the method. Preferably, the gas is introduced in an amount to provide the cells in the cell culture with sufficient oxygen and/or carbon dioxide. Additionally, gas may be withdrawn from the bag so as to remove gaseous waste and to keep the volume and pressure in the bag in a desired range, preferably at a constant level.

Introduction of medium/nutrients and/or gas into the bag may be done constantly, periodically or in amounts and at times when necessary or desired during the method of cultivating cells. Preferably, the temperature of the cell culture is held in a desired range, preferably at a constant level, during the method, or it may be varied as needed during the method. The temperature should be adjusted as suitable for cell viability, growth rate, cellular production, by-products and stability of desired cellular products. The suitable temperature varies depending on the cells used and the cellular product of interest.

In preferred embodiments, the cell culture is agitated. To this end, the culturing bag is placed onto a platform which is capable of moving the bag in a seesaw or rocking motion, thereby inducing waves in the cell culture. Thus, the cells are preferably cultured using the “wave technology”. A platform as described above having the specific features described above may be used. Preferably, the degree and rate of the motion of the platform is adjusted so as to provide sufficient mixing of the cell culture, in particular sufficient distribution of oxygen. However, the degree and rate of the motion of the platform should preferably be low enough so that the cells in the cell culture and/or the culturing bag are not damaged. In particular, the motion of the platform should preferably be adjusted so that the first outlet of the bag does not contact air during the motion, thus preventing it from drawing air.

Preferably, the cells are cultures in the bag using one or more of the following culturing conditions: temperature in the range of 10 to 50° C., preferably 15 to 40° C., more preferably 35 to 39° C., most preferably about 37° C.; oxygen saturation in the range of 10 to 60% DOT (dissolved oxygen tension), preferably 20 to 50%, more preferably 35 to 45%, most preferably about 40%; pH value in the range of 5 to 9, preferably 6.5 to 8, more preferably 7.0 to 7.5, most preferably about 7.2; perfusion rate in the range of 0.1 to 5 v/d (volumes/day), preferably 0.2 to 3 v/d, more preferably a varying perfusion rate starting with about 0.5 v/d and increasing up to 2 v/d. However, the optimal culturing conditions are dependent on the cells and medium used and the cellular product to be obtained. The skilled person is readily capable of identifying and selecting suitable culturing conditions for a given cell culture.

In a preferred embodiment, the method for cultivating cells according to the invention has all features described in claims 7 and 8 (i) to (xi).

c) The Method for Producing a Biological Substance

In a third aspect, the present invention provides a method for producing a biological substance comprising the following steps:
a) providing a culture of cells capable of producing the biological substance in a bag;
b) transferring a part of the cell culture from the bag to a continuous flow centrifuge;
c) separating said part of the cell culture into a first fluid of decreased cell density and a second fluid of increased cell density by continuous flow centrifugation; and
d) obtaining the biological substance from the first and/or second fluid; and
e) returning at least part of the first and/or the second fluid to the cell culture in the bag.

According to one embodiment, wherein the biological substance is secreted by the cells, the biological substance is obtained from the first fluid which is produced in step (c) and wherein at least a part of the second fluid obtained in step (c) is returned to the cell culture in the bag.

According to a further embodiment, wherein the biological substance is not secreted by the cells, at least a part of the second fluid which is obtained in step (c) and which contains the biological substance is collected to obtain the biological substance therefrom by conventional isolation and/or purification methods. Furthermore, at least a part of the remaining second fluid, if any, and/or at least part of the first fluid is returned to the cell culture in the bag.

The following embodiments in particular refer to the preferred embodiment wherein the biological substance is obtained from the first fluid and at least a part of the second fluid, if any, is recycled to the cell culture in the culturing bag. However, the described embodiments and features may also apply, as appropriate, to other embodiments wherein, for example, at least a part of the first fluid is recycled to the cell culture in the culturing bag, or wherein neither the first nor the second fluid are recycled to the cell culture in the culturing bag. Suitable modifications of said embodiments and features which are necessary to match these other embodiments of the method for culturing cells according to the invention are included herein and can be readily derived from the following description by a person skilled in the art.

Preferably, the cells producing the biological substance are cultivated according to the method for cultivating cells of the second aspect of the invention. Any and all embodiments described above may also be used in the method for producing a biological substance according to the invention.

The biological substance to be produced may be any substance that can be produced by a cell. The biological substances may be, for example, a peptide, protein, nucleic acid, lipid, amino acid, carbohydrate, virus, or part of a virus such as viral proteins and components or virus like-particles. Particular examples of biological substances are therapeutically active substances, immunoglobulins as well as antibodies including functional fragments or variants thereof, hormones, and toxins. Other specific examples of biological substances are growth factors, blood factors, cytokines, interleukines, interferons, tumor necrosis factors, gonadotrophins hormones, receptors, adhesion molecules (membrane-bound or secreted form), fusion proteins (e.g. antibody-fusion proteins), bi- or tri-specific antibodies, multiple antibodies, therapeutically useful viruses or parts thereof such as virus-like particles, in particular viruses or parts thereof useful for vaccination and/or vaccinia viruses or adenoviruses. Preferably, the biological substance is a peptide or protein, more preferably a glycosylated peptide or protein such as a glycosylated antibody, a glycosylated enzyme or a glycosylated receptor, or a part thereof.

If the biological substance to be produced is a virus or a part thereof or is encoded by a gene which is introduced into the cells via viral infection, the cells of the cell culture may be infected by the respective virus or part thereof before they are introduced into the culturing bag or they may be infected when they are already present in the bag, for example during the cultivation of the cells inside the bag. In particular, the cells may be infected when the cell culture has reached a specific cell density. For infection inside the culturing bag, infectious viruses or parts thereof may be introduced into the culturing bag in an amount suitable for infecting some of the cells of the cell culture, in particular in an amount which gives rise to an appropriate cellular production of the biological substance of interest after a suitable initiation period.

In particular embodiments, the biological substance produced by the cells is a virus or a part thereof. For example, it may be a life, infectious virus, an attenuated virus such as a virus which is less viable and/or less infectious than the virus from which it is derived, or a non-infectious virus. A virus may be rendered attenuated or non-infectious by introducing one or more mutations into the genome of the virus. Furthermore, the biological product may be a part of a virus such as a virus-like particle or a protein, in particular a glycoprotein of the virus. Preferably, the part of the virus exhibits one or more epitopes which are also present on the complete virus, in particular recognizable on the complete virus by a host's immune system. The part of the virus, in particular the virus-like particle may be infectious, but preferably is not infectious. In particular, the virus or part thereof is useful for vaccination. In a further embodiment, the virus or part thereof produced in the method according to the invention is useful in gene therapy. In particular, the virus or part thereof is infectious for mammalian cells, especially for human cells such as specific human cells of a certain tissue. In this embodiment, the virus or part thereof may carry a gene which can be integrated in the genome of the target cell. In particular, the gene is a therapeutic gene which is used for treating a disease in a patient, in particular a genetic disease such as an inherited disease or a disease caused by genetic mutations such as cancer. In the above embodiments, the virus or part thereof may in particular be capable of infecting a target cell, but may not be capable of producing further infectious viruses or parts thereof after infection of the target cell.

In one embodiment, the biological substance is secreted by the cells. In this case, the biological substance is mainly present in the medium of the cell culture. By collecting the first fluid obtained after centrifugation, the biological substance of interest is also collected. The biological substance preferably is then obtained respectively isolated from said collected first fluid. The biological substance may be isolated from the first fluid by any isolation method known in the art. However, the methods for isolating the biological substance may depend on the nature of the biological substance. The skilled person can readily identify and select the isolation methods suitable for any given biological substance. Preferably, the isolation methods include, for example, chromatographic methods, electrophoretic methods, precipitation methods and/or membrane absorption methods. The respectively isolated substance can be further processed, e.g. purified.

In another embodiment, the biological substance is not secreted by the cells. In this case, the biological substance of interest is mainly present inside or attached to the cells. By collecting all or a part of the second fluid obtained after centrifugation, the cells containing the biological substance and thus, also the biological substance itself is collected. Preferably, the biological substance is then isolated form the second fluid. For isolating the biological substance, the cells in the fluid are preferably first lysed or disrupted, for example by enzymatic and/or chemical lysis and/or mechanical or ultrasonic disruption, optionally after performing a washing step. Then cell debris may be separated from the fluid, for example by centrifugation, sedimentation or precipitation. The biological substance of interest may thereafter be isolated as described above for the secreted product. The first fluid obtained after centrifugation preferably is removed from the system in this embodiment but may also be returned to the bag if desired.

To maintain the cells in the growth phase and/or at a desirable cell density, also in the method for producing a biological substance the bleeding technique may be used. If the biological substance is secreted by the cells, bleeding may be performed as described above. However, if the biological substance is not secreted by the cells and at least a part of the cell-containing second fluid obtained after centrifugation is collected for obtaining the biological substance, said collection of the second fluid may effect the bleeding and thus, replace the removal of said second fluid done for bleeding the cell culture.

In a preferred embodiment, the method for producing a biological substance according to the invention has all features described in claims 9, 10, 12 (i) to (xii) and 13 or all features described in claims 9, 11, 12 (i) to (xii) and 14.

d) The Use of a Bag for Cultivating Cells

In a fourth aspect, the present invention provides the use of a bag comprising at least one outlet and at least one inlet for cultivating cells in a device according to the first aspect of the invention or in a method according to the second or third aspect of the invention.

The bag is preferably designed and has the features described above with respect to the system for culturing cells according to the first aspect of the invention and with respect to the methods according to the second and third aspects of the invention. In particularly preferred embodiments, the bag comprises the following features:
an outlet which is located at the bottom of the bag or at the side of the bag near the bottom or which comprises a tube extending to the bottom of the bag, useful for withdrawing cell culture from the bag;
an inlet useful for introducing fluids into the bag;
optionally and preferably a further inlet and a further outlet for introducing and withdrawing gas, optionally equipped with filters;
optionally and preferably one or more barriers attached to the bottom of the bag and/or protruding from the bottom of the bag and dividing the bottom of the bag into two or more separate basins;
optionally and preferably means for attaching the bag to a platform which moves the bag in a seesaw or rocking motion.

Preferably, the bag is flexible or rigid, preferably it is collapsible. It preferably is a plastic bag, more preferably a thermoplastic bag. The bag may be sterilized prior to its use. In preferred embodiments, the bag has only one single hollow interior space and/or does not contain a membrane and/or does not contain a stirring device.

In a preferred embodiment, the bag used according to the invention has all features described in claim 16 (i) to (iv) and (vii) to (xiv).

### FIGURES

FIG. 1 is a schematic, simplified drawing and shows the assembly of a preferred embodiment of the device for culturing cells according to the invention. Culturing bag 11 containing a cell culture 12 is placed upon a platform 13 for agitating the cell culture in a wave motion. Bag 11 further comprises a first outlet 14 for withdrawing cell culture. Outlet 14 is connected to a continuous flow centrifuge 17. In centrifuge 17, a part of cell culture 12 is separated in a first fluid of decreased cell density, leaving the centrifuge through a first outlet 18, and a second fluid of increased cell density. A second outlet of centrifuge 17 is connected with a first inlet 15 of bag 11 for recycling and thus returning at least a portion of the second fluid obtained after centrifugation into bag 11. Bag 11 is connected with centrifuge 17 via tubing 16. FIG. 1 is merely a schematic drawing. Thus, many variations of the shown design are possible. E.g. the tube 16 and the outlet 14 as well as the tube 16 and the inlet 15 are presented as one piece. However, they may of course also be formed by two or more separate but connected pieces.

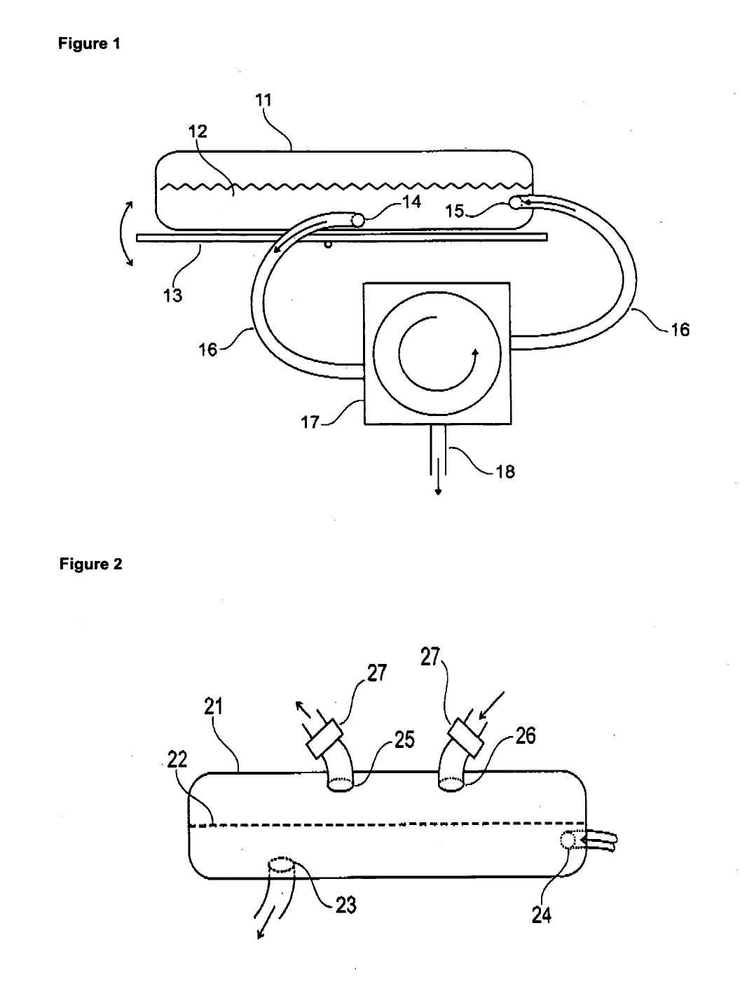

FIG. 2 shows a preferred embodiment of the culturing bag. Culturing bag 21 comprises a first outlet 23 for withdrawing cell culture. First outlet 23 is located at or near the bottom of bag 21, beneath the surface 22 of a cell culture filled into bag 21. Bag 21 further comprises a first inlet 24 for introducing cell-containing fluid into bag 21. Furthermore, bag 21 comprises a second inlet 26 and a second outlet 25 for introducing and withdrawing gas, respectively. Inlet 26 and outlet 25 are equipped with filters 27 for preventing the cell culture from being contaminated and from contaminating the environment.

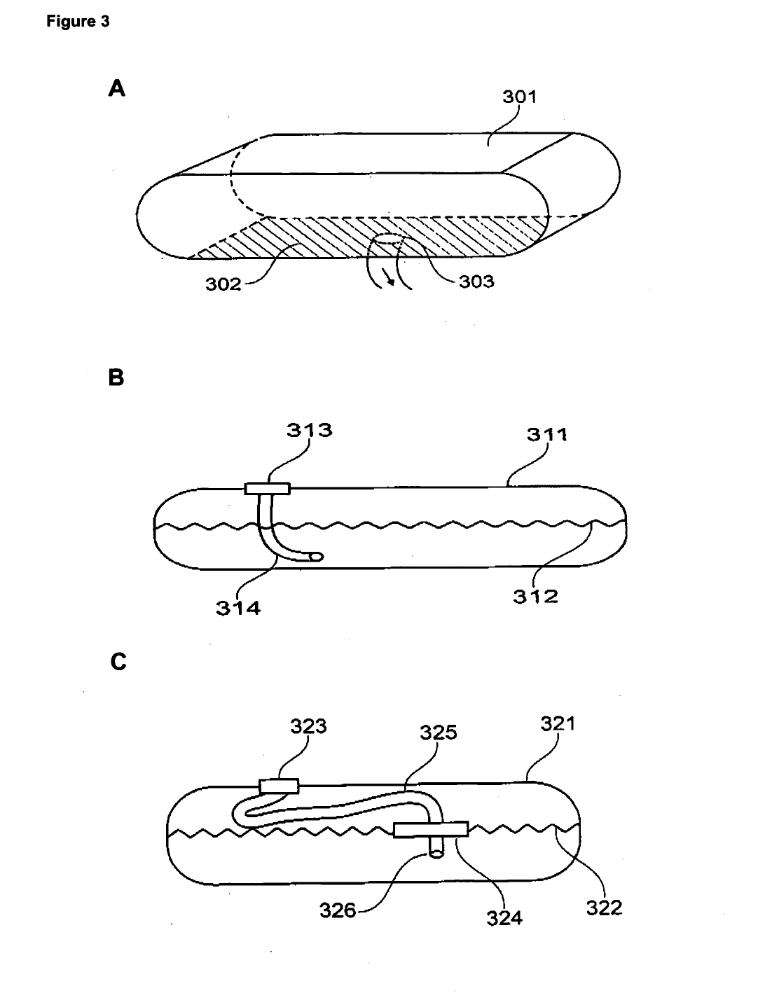

FIG. 3 shows various assemblies of the first outlet of the culturing bag. In FIG. 3A, first outlet 303 is located at the bottom 302 of bag 301. In FIG. 3B, first outlet 313 comprises a tube 314 extending into the interior of bag 311 beneath the surface 312 of the cell culture. In FIG. 3C, first outlet 323 comprises a tube 325 connected to a floating device 324 which floats on the surface 322 of the cell culture. A further tube 326 attached to floating device 324 protrudes into the cell culture. In a variation, tubes 325 and 326 are provided as one piece and thus as one tube which extends through and/or is attached to a floating device 324.

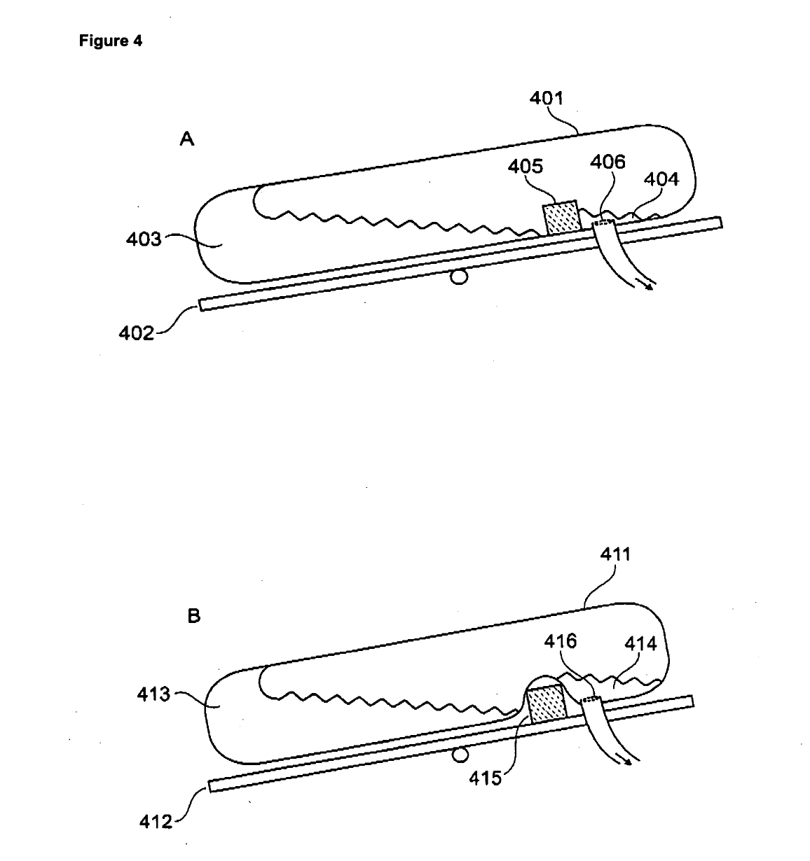

FIG. 4 shows various assemblies of the culturing bag and the device for culturing cells comprising a cell culture reservoir. In FIG. 4A, bag 401 is placed on platform 402 which moves bag 401 in a seesaw or rocking motion, introducing wave formation in cell culture 403. Barrier 405 attached to the bottom of bag 401 forms a separate basin in bag 401, thereby providing a cell culture reservoir with cell culture medium 404. Of course, the bag 401 and the barrier 405 may also be provided as one piece. First outlet 406 of bag 401 is located in the basin of cell culture reservoir comprising the cell culture medium 404. In FIG. 4B, bag 411 is placed on platform 412 which moves bag 411 in a seesaw or rocking motion, introducing wave formation in cell culture 413. Barrier 415 attached to platform 412 dents bag 411, thereby forming a separate basin in bag 411 providing the cell culture reservoir with cell culture medium 414. First outlet 416 of bag 411 is located in the basin of cell culture reservoir 414. Of course, the platform 412 and the barrier 415 may also be formed as one piece. In FIG. 4C, bag 421 is placed on platform 422 which moves bag 421 in a seesaw or rocking motion, introducing wave formation in cell culture 423. Outlet 424 of bag 421 is connected via tube 425 with vessel 426. Vessel 426 provides a cell culture reservoir with cell culture medium 427 and is connected via a tube 428 to continuous flow centrifuge 429. The outlet of vessel 426 is located beneath the surface of cell culture in reservoir 427. In FIG. 4D, bag 431 is placed on platform 432 which moves bag 431 in a seesaw or rocking motion, introducing wave formation in cell culture 433. Depression 435 formed in platform 432 allows bag 431 to form a basin containing cell culture in reservoir 434. First outlet 436 of bag 431 is located in the basin of cell culture reservoir 434.

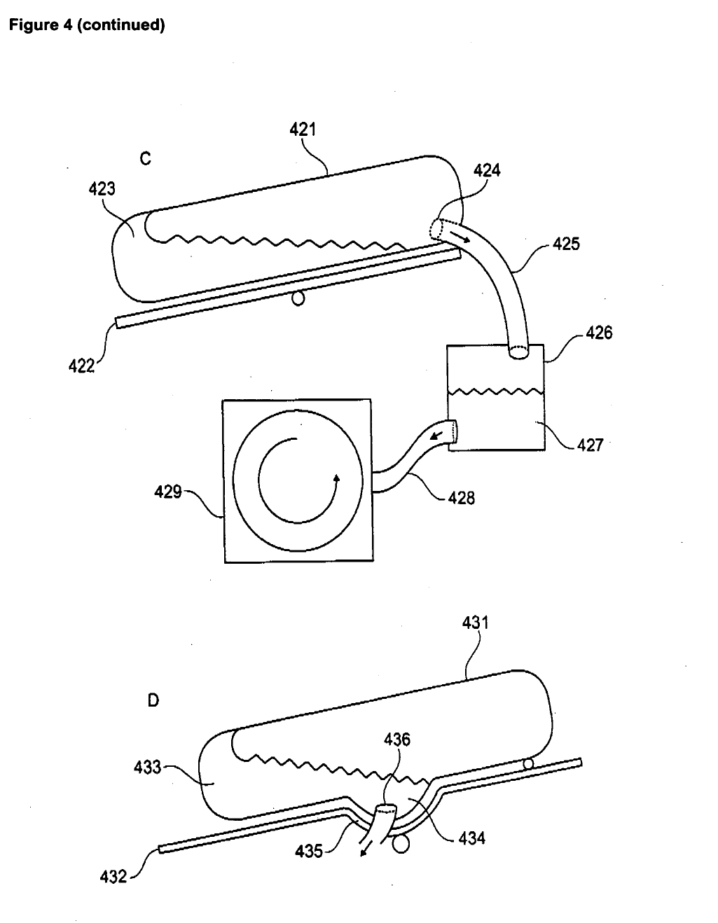

In these figures, some features of the bag or the system such as the inlet of the bag or the tubing connecting the centrifuge with the bag are not shown for clarity reasons.

FIG. 5 shows comparison data of the method according to the invention (W4) compared with a perfusion system for culturing cells using a floating filter device for withdrawing medium from the cell culture (W1). In both systems, human myeloid leukaemia derived cells expressing and secreting a monoclonal antibody were cultured under comparable conditions. A: viable cell density [cells/ml]; B: productivity [μg/ml]; C: glucose concentration [g/l], D: cell viability [%].

FIG. 6 shows comparison data of the method according to the invention (W4) compared with a stirred tank bioreactor (F2). In both systems, human myeloid leukaemia derived cells expressing and secreting a monoclonal antibody were cultured under comparable conditions. A: viable cell density [cells/ml]; B: productivity [μg/ml]; C: glucose concentration [g/l], D: cell viability [%].

FIG. 7 shows the relative amount of aggregated product for the method according to the invention (“Wave”) and a stirred tank bioreactor (“Stirred tank”). In both systems, human myeloid leukaemia derived cells expressing and secreting a monoclonal antibody were cultured under comparable conditions. The relative amount of non-monomeric antibodies is shown in percent of the entire amount of antibodies produced.

FIG. 8 shows the gylcoprofile of the product obtained by the method according to the invention (“Wave”) and a stirred tank bioreactor (“Stirred tank”). In both systems, human myeloid leukaemia derived cells expressing and secreting a monoclonal antibody were cultured under comparable conditions. The relative amount of oligosaccharides on the produced antibodies which carry the indicated glycosylation property are shown for both production methods. Bisect. GlcNAc: bisecting N-acetylglucosamine; S0: oligosaccharides without any sialic acids; S>0: oligosaccharides having at least one sialic acid; S1: oligosaccharides having one sialic acid; S2: oligosaccharides having two sialic acids; G0: oligosaccharides without any galactose units; G1: oligosaccharides having one galactose unit; G2: oligosaccharides having two galactose units.

### EXAMPLES

### 1. Exemplary Setup of the System Containing the Bag, the Platform and the Centrifuge

A modified wave cellbag disposable bioreactor having a volume of 0.5 l-5 l using a dip-tube for cell withdrawal was used. The bag comprised inlets for cell concentrate and base addition. The bag was placed onto a Wave Cellbase 20 SPS platform using a Wavepod for pH and DOT control. The movement of the platform was adjusted to an angle of 6° and 12 rocks/minute.

The bag was connected to a Centritech Lab II continuous flow centrifuge. Centrifugation was performed at 41*g. The pump rates for the feed pump and concentrate pump were adjusted to 4 l/h in an intermittent working mode.

### 2. Exemplary Method for Cultivating Cells and/or Producing a Secreted Product

In the culturing system according to example 1, a suspension cell line such as, e.g., CHO, NS0, K-562, or Glycoexpress was cultivated. To this end, serum-free or protein-free media (e.g. Ham's F12, DMEM, ProCHO5, X-Vivo20, X-Vivo15, ProDoma 3, Ex-Cell 302, Ex-Cell CHO) including Pluronic F-68 was provided in the culturing bag.

Preculturing was performed in T-flasks or spinner bottles. Human myeloid leukaemia derived cells expressing and secreting a monoclonal antibody were incubated at 37° C., 8% CO₂, and 90% humidity. The cells expanded with an initial seeding density of 1.5*10̂5 cells/ml. For the main culture, the bioreactor was inoculated with 1.5*10̂5 cells/ml at 10% of the total volume. The initial glucose concentration was at about 4 g/l. The cells were cultured at 37° C., pH 7.2, and 40% DOT.

Perfusion was started when the glucose concentration dropped under 2.5 g/l with a perfusion rate of 0.5 volumes/day. Perfusion was increased by 0.5 V/d, when the glucose concentration dropped below 1.5 g/l until the maximal perfusion rate of 2 V/d was reached.

Bleeding was applied in order to avoid glucose limitation at maximal perfusion rate. The bleeding rate was set between 1% and 10%. The time of cultivation in process development was 10-40 days.

### 3. Comparison Between the Methods According to the Invention and Prior Art Methods

3.1. Comparison to a Perfusion System with Floating Filter Apparatus

The perfusion system described in examples 1 and 2, above, was compared to a similar perfusion system wherein a floating filter setup instead of a continuous flow centrifuge was used. In the floating filter system, culture medium was withdrawn from the cell culture using a filter which floats on the surface of the cell culture and through which culture medium but not cells can be withdrawn from the cell culture.

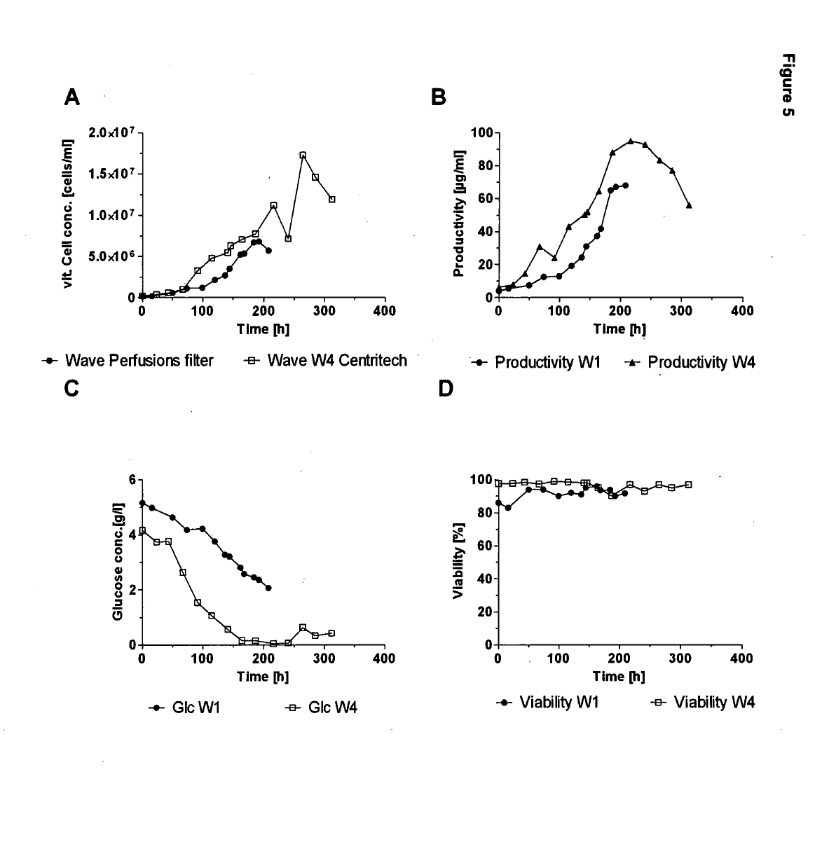

The results obtained using the method and system according to the invention and the cell cultivation with a floating filter perfusion system are shown in FIG. 5.

Under comparable culturing conditions, a significantly higher cell density could be reached using the method according to the invention. Along with this, a significantly improved productivity (about 50% increased yield of the secreted antibody in the medium) could be achieved. Furthermore, the higher glucose consumption rate observed with the method according to the invention also indicates better cell growth.

Both systems provided for a very high cell viability (>95%) indicating that the viability is not affected by the higher cell densities obtained in the method according to the invention.

Furthermore, the system according to the invention has the further advantages, compared to the floating filter perfusion system that it provides a much better scalability, in particular because continuous flow centrifuges having a higher flow-through and thus, being suitable for large scale industrial applications are available. In contrast, the size of the floating filter is limited and not freely adaptable for upscaling. In addition, the system according to the invention avoids the problem of membrane fouling and clogging.

3.2. Comparison to a Perfusion System with Fermenter (Not Wave Technology) and Continuous Flow Centrifuge

In a second comparison, the perfusion system described in examples 1 and 2, above, was compared to a similar perfusion system with a fermenter and a continuous flow centrifuge. In the fermenter system, the cell culture is mixed by a stirring device rather than using the wave technology.

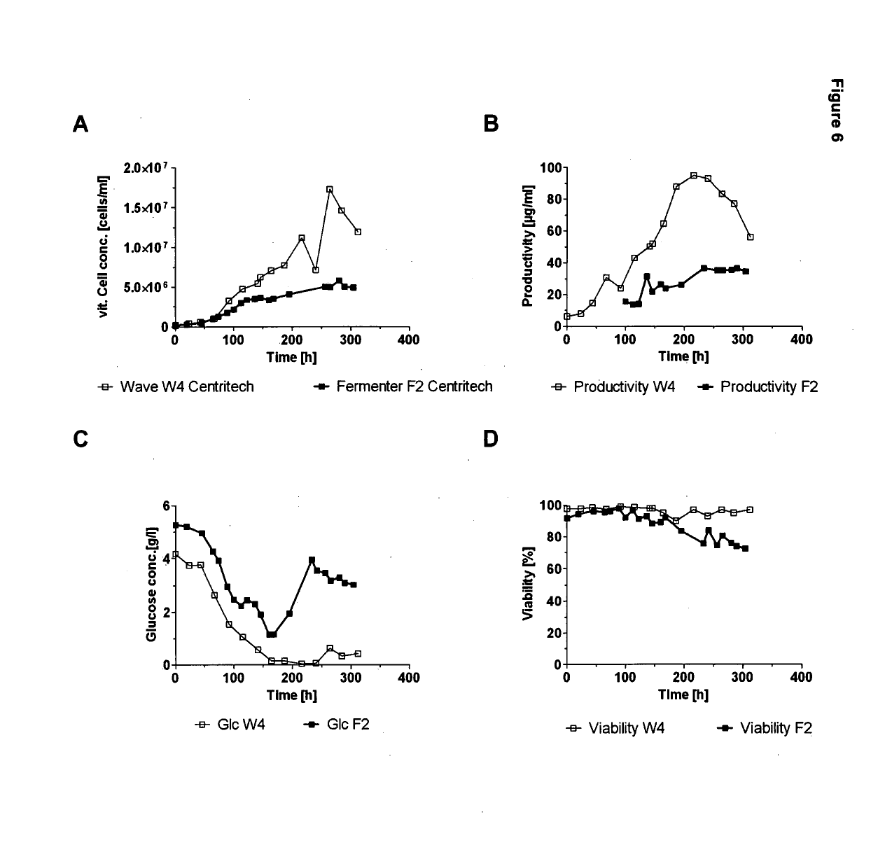

The results obtained using the method and system according to the invention and the fermenter system are shown in FIG. 6.

Under similar culturing conditions, a significantly increased cell density (+ about 100%) could be reached using the method according to the invention. Along with this, a significantly improved productivity (about 100% increased yield of the secreted antibody in the medium) could be achieved. Furthermore, higher cell viability was observed in the wave bioreactor according to the invention. The higher viability may be due to less shear forces in the wave bioreactor. The stirring in the conventional stirred tank reactor decreases viability due to shear stress at the stirring device. The wave culture remained longer at a high cell viability and there was no significant drop in viability. Therefore, a high glucose uptake and high cell densities are possible when using the system according to the invention.

Furthermore, compared to the fermenter system, the system according to the invention has the further advantages that it avoids costly and time consuming maintenance such as, in particular CIP/SIP (cleaning in place/sterilization in place). Rather, disposable bags can be used which can be quickly changed between different culturing runs. This also decreases the downtime of the system between the runs.

3.3. Comparison of the Obtained Protein Quality

In the experimental setup of example 3.2., also the stability and quality of the proteins obtained by the different culturing methods has been determined and compared to each other.

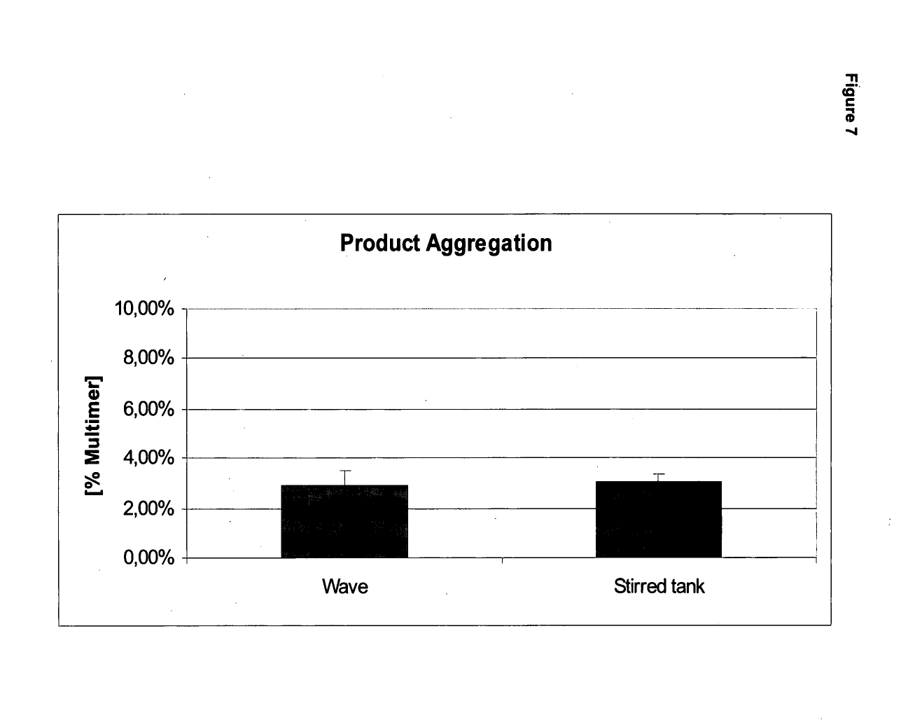

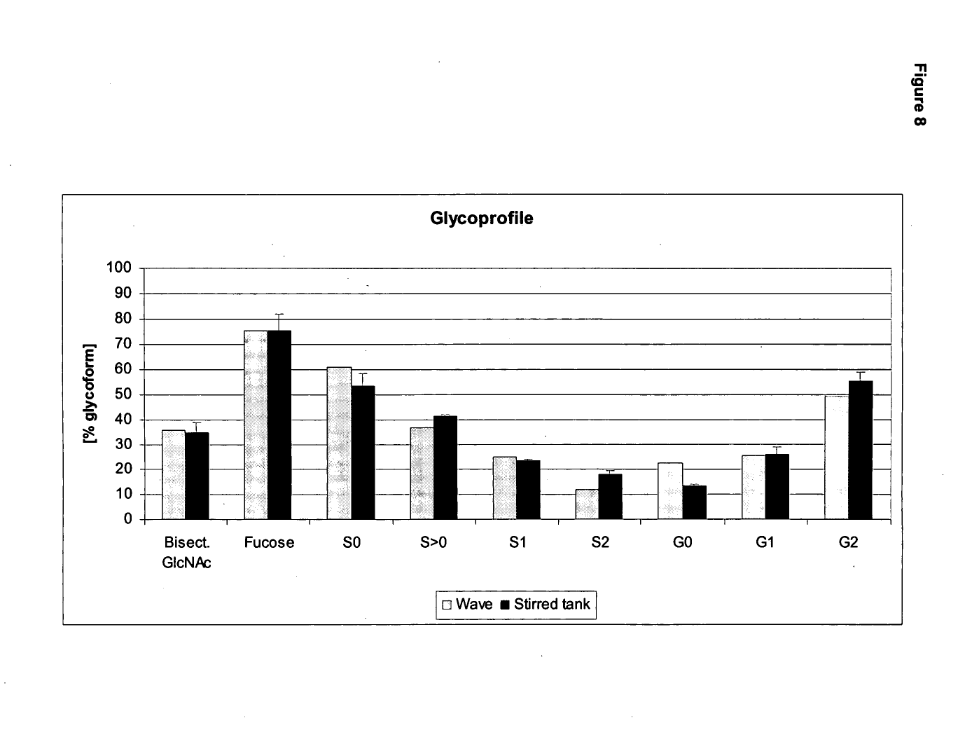

The results obtained in this experiment are shown in FIGS. 7 and 8.

It is demonstrated by these experiments that although the method and system according to the present invention provides much higher cell densities and a highly increased protein production, the stability and quality of the obtained proteins is similar to the protein quality obtained by the fermenter system. In particular, the relative amount of aggregated protein is identical for both methods, with the mean ratio of aggregated protein even being slightly lower for the system according to the present invention (2.9% aggregation compared to 3.1% aggregation). Furthermore, also the glycoprofile of the obtained glycoproteins is nearly identical for the different cultivation systems. Thus, both systems provide glycoproteins with similar quality.

This is a surprising and unexpected finding since the much higher production rate and protein concentration in the system according to the present invention are potentially detrimental to the stability and quality of the produced proteins. Thus, the method and system according to the present invention allows for a doubling of the productivity compared to conventional fermentation processes without any deterioration in protein quality and stability.

### DEPOSITED MATERIAL REFERRED TO HEREIN

This application refers to the following biological material:

| | Accession | depositary | Name and address | |
|---|---|---|---|---|
| Zell line | number | institution | of the depositor | Comments |
| NM-F9 | DSM ACC2606 | DSMZ¹ | Nemod | The cell line was |
| | | | Biotherapeutics | assigned from |
| | | | Robert-Rössle- | Nemod |
| | | | Straβe 10 | Biotherapeutics to |
| | | | 13125 Berlin | Glycotope GmbH |
| NM-D4 | DSM ACC2605 | DSMZ¹ | Nemod | The cell line was |
| | | | Biotherapeutics | assigned from |
| | | | Robert-Rössle- | Nemod |
| | | | Straβe 10 | Biotherapeutics to |
| | | | 13125 Berlin | Glycotope GmbH |
| NM-H9D8 | DSM ACC 2806 | DSMZ¹ | Glycotope GmbH | |
| | | | Robert-Rössle- | |
| | | | Straβe 10 | |
| | | | 13125 Berlin | |
| NM-H9D8-E6 | DSM ACC 2807 | DSMZ¹ | Glycotope GmbH | |
| | | | Robert-Rössle- | |
| | | | Straβe 10 | |
| | | | 13125 Berlin | |
| NM H9D8-E6Q12 | DSM ACC 2856 | DSMZ¹ | Glycotope GmbH | |
| | | | Robert-Rössle- | |
| | | | Straβe 10 | |
| | | | 13125 Berlin | |
| GT-2X | DSM ACC 2858 | DSMZ¹ | Glycotope GmbH | |
| | | | Robert-Rössle- | |
| | | | Straβe 10 | |
| | | | 13125 Berlin | |

¹DMSZ: Deutsche Sammlung von Mikroorganismen und Zellkulturen GmbH Inhoffenstr. 7B, 38124 Braunschweig, DE

Additional Indications According to Form PCT/RO/134 for Accession Number DSM ACC2606:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

Additional Indications According to Form PCT/RO/134 for Accession Number DSM ACC2605:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

Additional Indications According to Form PCT/RO/134 for Accession Number DSM ACC2806:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

Additional Indications According to Form PCT/RO/134 for Accession Number DSM ACC2807:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

Additional Indications According to Form PCT/RO/134 for Accession Number DSM ACC2856:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

Additional Indications According to Form PCT/RO/134 for Accession Number DSM ACC2858:

Applicant herewith requests for those countries which have a respective provision that the furnishing of a sample of the deposited material referred to in the application may only be made to an independent, nominated expert (request of the “expert solution” where applicable, in particular in Australia, Canada, Croatia, Denmark, Finland, Germany, Iceland, Norway, Singapore, Spain, Sweden, United Kingdom, Europe).

For Europe, applicant accordingly requests that a sample of the deposited biological material will be made available as provided in Rule 33(1)(2) EPC until the publication of the mention of the grant of the patent or for 20 years from the date of filing if the application is refused or withdrawn or deemed to be withdrawn, only by the issue of a sample to an expert nominated by the person requesting the sample (Rule 32 EPC).

1. A system for culturing cells, comprising:
a) a container for a cell culture comprising at least one outlet and at least one inlet;
b) a means for moving the container in a seesaw, rocking or rotational motion; and
c) a continuous flow centrifuge for separating cell culture into a first fluid of decreased cell density and a second fluid of increased cell density, the centrifuge comprising (a) at least one inlet, (b) a first outlet for the first fluid and (c) a second outlet for the second fluid.

2. The system according to claim 1, further comprising a means for returning at least a portion of the first or the second fluid to the cell culture after separation by the continuous flow centrifuge.

3. The system according to claim 1, wherein at least one outlet of the container is connected with at least one inlet of the centrifuge, and at least one outlet of the continuous flow centrifuge is connected with at least one inlet of the container.

4. The system according to claim 3, wherein the connection is directly or indirectly.

5. The system according to claim 1, wherein the cell culture container has one or more of the following characteristics:
(i) the first outlet of the container is located beneath the surface of the cell culture when present in the container;
(ii) the first outlet is located at the bottom of the container or comprises a tube extending into the cell culture;
(iii) the container comprises a bag;
(iv) the container comprises a plastic bag;
(v) the container comprises only one single hollow interior space;
(vi) the container further comprises an inlet for introducing gas containing oxygen and/or carbon dioxide, optionally equipped with a filter;
(vii) the container further comprises an outlet for withdrawing gas, optionally equipped with a filter;
(viii) the container further comprises an inlet for introducing media, nutrients or inoculum;
(ix) the container further comprises an outlet for withdrawing cell culture;
(x) the container does not comprise a membrane; and/or (xi) the container does not comprise a stirring device.

6. The system according to claims 1, further comprising:
(i) means for forming a cell culture reservoir for preventing the centrifuge from drawing air;
(ii) tubing which connects the container and the centrifuge;
(iii) a means for introducing cell culture components connected to at least one inlet of the container;
(iv) a means for introducing oxygen or carbon dioxide connected to the inlet of the container;
(v) a means for withdrawing at least a part of the second fluid from the continuous flow centrifuge, the means being connected to the outlet of the centrifuge or the tubing between the outlet of the centrifuge and the inlet of the container;
(vi) a heating device;
(vii) one or more sensors for measuring a parameter selected from the group consisting of the cell density in the cell culture, the second fluid, the first fluid, the concentration of nutrients, the oxygen level, the carbon dioxide level, the cell products, by-products, toxins, cell degradation products, the pH value in the cell culture, the concentration of oxygen or carbon dioxide in the air in the container, the concentration of cell products in the first fluid, the temperature of the cell culture, and combinations thereof;
(viii) at least one adapter for connecting the outlet of the container to the inlet of the continuous flow centrifuge or the outlet of the continuous flow centrifuge to the inlet of the container; or
(ix) more than one continuous flow centrifuge connected to the container.

7. A method for cultivating cells comprising the steps of:
a) providing a cell culture in a container;
b) transferring a part of the cell culture from the container to a continuous flow centrifuge;
c) separating said part of the cell culture into a first fluid of decreased cell density and a second fluid of increased cell density by continuous flow centrifugation; and
d) returning at least a part of the first or second fluid to the cell culture in the container;
wherein the cell culture is agitated by moving the container comprising the cell culture in a seesaw, rocking or rotational motion.

8. The method according to claim 7,:
(i) the part of the cell culture is transferred continuously or periodically from the container to the continuous flow centrifuge;
(ii) the part of the cell culture is transferred without transferring a substantial amount of gas to the centrifuge;
(iii) the first fluid obtained after centrifugation or a part thereof is removed from the system;
(iv) medium or nutrients are continuously or periodically added to the cell culture in the container, preferably in an amount similar to the amount of the first fluid withdrawn from the system and/or in an amount to provide the cells of the cell culture with sufficient fresh medium or nutrients;
(v) gas containing oxygen or carbon dioxide is introduced into the container, preferably in an amount to provide the cells of the cell culture with sufficient oxygen and/or carbon dioxide;
(vi) a part of the second fluid is removed from the system, preferably in an amount sufficient to maintain the cells in the cell culture in the growth phase and/or in an amount sufficient to maintain the cell density in the cell culture at a desired, in particular constant level;
(vii) the temperature of the cell culture is maintained at a constant temperature suitable for cell viability and growth;
(viii) the container is a bag;
(ix) the cells are eukaryotic or prokaryotic, preferably bacteria, yeast, or animal cells;
(x) the container comprising the cell culture is moved in a seesaw, rocking or rotational motion by a platform onto which the bag is placed;
(xi) the first fluid of decreased cell density is substantially cell-free.

9. A method for producing a biological substance comprising:
a) providing a culture of cells capable of producing the biological substance in a container;
b) transferring a part of the cell culture from the container to a continuous flow centrifuge;
c) separating said part of the cell culture into a first fluid of decreased cell density and a second fluid of increased cell density by continuous flow centrifugation; and
d) obtaining the biological substance from the first and/or second fluid; and
e) returning at least part of the first and/or the second fluid to the cell culture in the container
wherein the cell culture is agitated by moving the container comprising the cell culture in a seesaw, rocking or rotational motion.

10. The method according to claim 9, wherein the biological substance is secreted by the cells and wherein the biological substance is obtained from the first fluid which is produced in step (c) and wherein at least a part of the second fluid obtained in step (c) is returned to the cell culture in the container.

11. The method according to claim 9, wherein the biological substance is not secreted by the cells, and wherein at least a part of the second fluid which is obtained in step (c) and which contains the biological substance is collected, and wherein at least a part of the remaining second fluid, if any, or at least part of the first fluid is returned to the cell culture in the container.

12. The method according to claim 9, wherein the method has one or more of the following characteristics:
(i) the part of the cell culture is transferred continuously or periodically from the container to the continuous flow centrifuge;
(ii) the part of the cell culture is transferred without transferring a substantial amount of gas to the centrifuge;
(iii) medium or nutrients are continuously or periodically added to the cell culture in the container, preferably in an amount similar to the amount of the first fluid withdrawn from the system or in an amount to provide the cells of the cell culture with sufficient fresh medium or nutrients;
(iv) gas containing oxygen or carbon dioxide is introduced into the container, preferably in an amount to provide the cells of the cell culture with sufficient oxygen or carbon dioxide;
(v) the temperature of the cell culture is maintained at a constant temperature suitable for cell viability and growth;
(vi) the container is rigid a bag;
(vii) the cells are grown in suspension;
(viii) the cells are eukaryotic or prokaryotic, preferably bacteria, yeast, or animal cells;
(ix) the biological substance is a peptide, a protein, a nucleic acid, a virus or virus-like particle or an organic compound, an immunoglobulin, an antibody or a binding variant or fragment thereof, a growth factor, cytokine, hormone or a toxin;
(x) a part of the second fluid is removed from the system, preferably in an amount sufficient to maintain the cells in the growth phase or in an amount sufficient to maintain the cell density in the cell culture at a desired, in particular constant level;
(xi) the container comprising the cell culture is moved in a seesaw, rocking or rotational motion by a platform onto which the bag is placed; or (xii) the first fluid of decreased cell density is substantially cell-free.

13. The method according to claim 9, wherein the biological substance is secreted by the cells and wherein:
(i) the biological substance is isolated from the first fluid;
(ii) the biological substance is isolated from the first fluid using chromatographic methods; or
(iii) the biological substance is further processed.

14. The method according to claim 9, wherein the biological substance is not secreted by the cells and wherein:
(i) the biological substance is isolated from the second fluid;
(ii) the biological substance is isolated from the second fluid by lysing or disrupting the cells, preferably by separating cellular debris from the biological substance and using chromatographic methods;
(iii) the biological substance is further processed; or
(iv) the first fluid or a part thereof is removed from the system.

15. Use of a container comprising at least one outlet and at least one inlet for cultivating cells in a system according to claim 1 or in a method according to claim 7 or 9.

16. The use according to claim 15, wherein the:
(i) is flexible or rigid;
(ii) is a plastic bag;
(iii) is sterilized prior to use;
(iv) at least one outlet is formed so that it is located beneath the surface of a cell culture to be placed inside the container;
(v) at least one outlet is located at the bottom of the container
(vi) at least one outlet comprises a tube extending into a cell culture to be placed inside the container;
(vii) contains a further outlet and a further inlet for introducing and withdrawing gas, respectively, optionally equipped with a filter;
(viii) has only one single hollow interior space;
(ix) has a further inlet for introducing media, nutrients, or inoculum;
(x) has a further outlet for withdrawing cell culture;
(xi) does not contain a membrane;
(xii) does not contain a stirring device;
(xiii) further comprises one or more barriers which are attached to the bottom of the container and separate the bottom of the container into two or more separate basins; and/or
(xiv) further comprises means for moving the container in a seesaw, rocking or rotational motion.

17. The use according to claim 15 for producing a biological substance of interest.

18. The system of claim 1, wherein the means for moving the container in a seesaw, rocking or rotational motion is a platform.

19. The system of claim 3, wherein the at least one outlet of the continuous flow centrifuge is the second outlet.

20. The system of claim 6, wherein the cell culture reservoir is equipped with a sensor for detecting the presence of cell culture in the reservoir.
